(19) United States
(12) Patent Application Publication
(10) Pub. No.: US 2020/0405856 A1
(43) Pub. Date: Dec. 31, 2020

(54) SONO-RESPONSIVE EMBOLIC AGENTS

(71) Applicant: IMBIOTECHNOLOGIES LTD., Edmonton (CA)

(72) Inventors: Michael STEWART, Edmonton, Alberta (CA); Irwin GRIFFITH, Edmonton, Alberta (CA)

(21) Appl. No.: 16/644,142

(22) PCT Filed: Sep. 7, 2018
(86) PCT No.: PCT/CA2018/051092

§ 371 Date: Mar. 3, 2020

Related U.S. Application Data
(60) Provisional application No. 62/555,430, filed on Sep. 7, 2017.

(51) Int. Cl. A61K 41/00; A61L 24/04; A61L 24/10; A61K 49/22

(52) U.S. Cl. CPC A61K 41/0028; A61L 24/046; A61L 2430/36; A61K 49/225; A61L 24/102

(57) ABSTRACT

The present invention relates generally to treatment methods using sono-responsive embolic agents and ultrasound to monitor the location or travel of the embolic agents. The embolic agents may be modified to alter wettability for enhanced treatment of various disease states.

### FIELD OF THE INVENTION

The present invention relates generally to the use of sono-responsive particles as therapeutic agents, particularly as embolic agents.

### BACKGROUND

Ultrasound is used as a minimally-invasive means of imaging structures within a solid body. Sound emitted at high frequency from a source encounters surfaces within a solid body that reflect waves back to the source. These reflected sound waves are converted to electronic signals and can be manipulated to provide images of the structures within a solid body. These images can be displayed as 2-dimensional, 3-dimensional, or 4-dimensional renderings. A key advantage of ultrasound is the minimally-invasive nature of the technology as it relates to use in medical and other applications requiring precision and minimal disturbance to and effect on the structures analyzed.

Ultrasound has been used as a method of monitoring fetal development and assessment of body structures such as organs including bladder, kidney, heart, spleen, gallbladder, pancreas, ovaries, uterus, thyroid, stomach, lungs, adrenals, prostate, and other structures including brain, eyes, blood vessels, lymphatic vessels, testicles, breast, and gastro-intestinal tract.

Contrast agents work with to ultrasound improve visualization of the vasculature system and the movement of blood to, within, and from structures inside the body. These ultrasound contrast agents consist of small structures encapsulating a gas, for example, nitrogen contained within a gelatin membrane. Ultrasound contrast agents serve to reflect ultrasound waves back to the source enabling real-time visualization of a tissue and its vasculature.

This background information is provided merely to provide information believed to be relevant to a basic understanding of the present invention. It is not an admission that any of the foregoing is prior art against any aspect of the claimed invention.

### SUMMARY OF THE INVENTION

In one aspect, the invention may comprise a method of treating a patient in need of embolic treatment, comprising the steps of:
(a) introducing a sono-responsive embolic agent into the patient; and
(b) monitoring the travel and/or position of the sono-responsive embolic agent by ultrasound.

In another aspect, the invention may comprise a method of monitoring a treatment involving the use of a sono-responsive embolic agent comprising the step of determining the travel, location and/or degradation of the sono-responsive embolic agent by ultrasound.

### BRIEF DESCRIPTION OF THE DRAWINGS

FIG. 1 shows an ultrasound sonogram of a uterine fibroid, prior to treatment with a sono-responsive embolic agent.

FIG. 2 is a sonogram of the same uterine fibroid 24 hours post-embolization.

FIG. 3 is a sonogram of the same uterine fibroid taken 30 days post-embolization.

### DETAILED DESCRIPTION

The present invention relates to compositions comprising a material with sono-responsive properties to reduce or eliminate the blood supply to or from a target tissue, along with methods of manufacturing and using such compositions. These compositions can be administered to an organism with a vascular supply to achieve a therapeutic benefit.

As used herein, the term “sono-responsive” refers to hyperechoic or hypoechoic properties of a material. A hyperechoic material strongly reflects ultrasound waves, while hypoechoic material poorly reflects ultrasound waves. The magnitude of hyperechogenicity or hypoechogenicity is not as important as the difference between the structure of interest and a surrounding structure. A sono-responsive material will provide sufficient contrast so as to be detectable or visible on a sonogram due to its hyperechoic or hypoechoic contrast with surrounding tissues or structures.

Sono-responsive embolic agents provide hyperechoic or hypoechoic contrast in vivo using ultrasound diagnostic methods. Such ultrasound methods typically employ frequencies in the range of about 2 MHz to about 15 MHz. Because higher frequencies of ultrasound have shorter wavelengths and are absorbed/attenuated more easily, they are not as penetrating. Thus higher frequencies are used for more superficial body structures and more penetrating lower frequencies are used for those that are deeper. For example, deep abdomen and obstetric imaging may use frequencies in the 2.5 to 3.5 MHz range.

The sono-responsive embolic agents have the property of acting as, producing, or facilitating the formation of an embolus. An embolus is a body of material which travels through the blood stream until it reaches a vessel which is too small to let it pass. The embolus then blocks blood flow in that vessel.

These sono-responsive embolic agents can be formulated in different manners and administered to an organism or placed within the organism using typical methods of delivery including, but not limited to, delivery by syringe, delivery by syringe and needle, delivery by catheter, or surgical delivery.

The sono-responsive embolic agents described herein may bind, or be altered or modified to bind blood cells, including platelets. Blood cells adhere to materials that have specific chemical groups and combinations of chemical groups on the surface of the material. Further platelets and blood cells adhere to materials with varying degrees of wettability. A wettable surface demonstrates an ability to maintain contact with a liquid. The degree of wetting or wettability is determined by a force balance between adhesive and cohesive forces.

Platelets function in the body to limit bleeding after blood vessels have been damaged. Initially, platelets adhere to components of the extracellular matrix and subendothelium. The adherent platelets are activated facilitating the binding of proteins circulating in the blood stream that function to further activate the platelets and act as bridges between platelets. These platelet clumps are held together by the end product of the coagulation cascade, fibrin, resulting in a blood clot. Platelet adhesion is essential in maintaining hemostasis.

In one embodiment, the sono-responsive embolic agents are used to maintain hemostasis in response to vascular trauma. Vascular trauma includes, but is not limited to, lacerations, contusions, surgical intervention, puncturing a blood vessel(s), disrupting the integrity of a blood vessel by chemical or physical means, and blunt trauma. Results of vascular trauma include, but are not limited to, excessive blood loss, minor blood loss, bruising, formation of petechiae, hemorrhage, hemorrhagic stroke, exsanguination, tissue damage, organ damage, nerve damage, muscle damage, damage to bones and joints.

In another embodiment, the sono-responsive embolic agents are used to induce thrombosis in the blood vessels or sinusoids of hypervascular tumors. In another embodiment, the materials are used in the treatment of aneurysms, arteriovenous malformations, endoleaks, varicoceles, peripheral vascular disease, benign prostatic hyperplasia, obesity, or incisions and puncture wounds. The materials may also be used to treat damaged blood vessels, regardless of the source of damage. For example, blood vessels may be damaged by medical devices such as guide wires, catheters, vascular access devices, drains or other agents disrupting the integrity of the blood vessel, or damaged by medical procedures involving high intensity ultrasound, radiofrequency ablation, irradiation, thermal ablation, cryoablation, laser ablation, microwave ablation, chemotherapy, radiation therapy, sclerosing agents, toxins, venoms, cadherin analogues or mimetics, or agents that disrupt the integrity of the affected blood vessel(s), increases or decreases in air pressure.

In other embodiments, the sono-responsive embolic agents are used in combination with other therapies used in the treatment of vascular trauma, hypervascular tumors, hyperplastic tissue, hypertrophic tissue, obesity, arteriovenous malformations, aneurysms, endoleaks, peripheral vascular disease and varicoceles.

In some embodiments, the sono-responsive embolic agents comprise glass, ceramics, polymers, metals, alloys, elastomers, pyrolytic carbon and plastics, or combinations thereof. Exemplary polymers include polyvinyl alcohol (PVA); polyglycol; polyglyconate; polyetheretherketone; polyacetal; polystyrene; polycarbonate; polylactide; polyglycolide; lactide-glycolide copolymers; polycaprolactone; lactide-caprolactone copolymers; hydroxyapatite; polyhydroxybutyrate; polyalkylcyanoacrylates; polyanhydrides; polyorthoesters; polysaccharides; dextrans; starches; methyl methacrylate; methacrylic acid; hydroxylalkyl acrylates; hydroxylapatite; hydroxylalkyl methacrylates; methylene glycol dimethacrylate; acrylamide; bisacrylamide; cellulose-based polymers; polyethylene; polyethylene terephthalate; ethylene glycol polymers and copolymers; oxyethylene and oxypropylene polymers; trimethylenecarbonate; polyvinyl acetate; polyvinylpyrrolidone and polyvinylpyridine; alone or in combination.

Generally, materials which have a density materially different than water (1.0 g/ml) will be sono-responsive. Hyperechoic materials will generally have a greater density than water. Therefore, in one embodiment, the sono-responsive material has a density greater than about 1.1 g/ml, preferably greater than about 1.2 g/ml, and more preferably greater than about 1.3 g/ml. In a like manner, hypoechoic materials will generally have a density less than water. Therefore, in another embodiment, the sono-responsive material has a density less than about 0.95 g/ml, preferably less than about 0.90 g/ml, and more preferably less than about 0.85 g/ml.

Materials which are not sono-responsive can be rendered sono-responsive by combination with other materials, or by alteration or modification. Methods of enhancing the sono-responsive nature of a material or combination of materials include, but are not limited to, facilitating attachment of microbubbles to the surface of the material (Takalar et al, 2004; Binding and detachment dynamics of microbubbles targeted to P-selectin under controlled shear flow. Journal of Controlled Release 96:473-482).

In preferred embodiments, the material comprises a hyperechoic or hypoechoic polymer. Preferably, the polymer comprises an ester comprising lactic acid, glycolic acid, caprolactone, hydroxybutyrate, and co-polymers of lactic acid, glycolic acid, caprolactone, or hydroxybutyrate. In some embodiments, the material may optionally be fully or partially biodegradable.

In some embodiments, the material may be constituted in a particle, either alone or in combination with other active or inactive materials. For example, poly(lactic-co-glycolic acid) (PLGA) particles may be conveniently manufactured or are readily commercially available. PLGA has a density of about 1.3 g/ml and are strongly hyperechoic in vivo. Other exemplary polymers with densities of about 1.1 g/ml to 1.5 g/ml include polyethylene, poly(methyl methacrylate), and cellulose acetate. Hypoechoic agents may be formed by introducing gas bubbles into a material to reduce its density.

In some embodiments, the sono-responsive embolic agent may be modified or altered to allow or enhance binding of blood cells, including platelets. Certain hypoechoic or hyperechoic materials, such as metals and polymers, in their native states, minimally bind or do not bind blood cells. Upon modification of the material by chemical, temperature or physical means, during or after the manufacturing process, the material will bind, or exhibit enhanced binding of, blood cells while retaining the sono-responsive properties of the material. Materials modified in this manner are useful in inducing or improving clot formation through blood cell capture, or in binding blood cells to achieve hemostasis.

A modification that imparts blood cell binding properties includes, but is not limited to, introduction of active groups on the surface of the material to alter the wettability of the material. Examples of active groups include metal oxides, fluoro, carbonyl, hydroperoxide, methyl, amino, hydroxyl, and carboxyl moieties. The material may be modified to be more or less wettable, or enhance blood cells and platelets binding, by treatment with radiation, such as gamma radiation, a chemical agent, such as hydrogen peroxide, an inorganic or organic base (e.g. potassium hydroxide, sodium hydroxide or butylamine), an inorganic or organic acid (e.g. hydrofluoric acid, phosphoric acid, nitric acid, sulfuric acid, oxalic acid, stearic acid, propionic acid, or valeric acid), or a protein such as collagen.

In some embodiments, the material may be coated with collagen, which greatly enhances its ability to bind platelets.

Material wettability can be assessed by various means including, but are not limited to, measuring the contact angle of a liquid on the material surface, determining the hysteresis between the contact angle of a liquid applied to a material surface in comparison to the contact angle of removing a liquid from a material surface, and calculating the spreading coefficient of a liquid on the material surface, and passage of a particle through a wettable or non-wettable conduit. Materials demonstrating wettability within a certain range have a greater propensity to bind blood cells such as platelets.

Platelet binding to a material can be assessed by various techniques including macroscopic examination, microscopic examination, identification of bound platelets using platelet-specific antibodies and chemical analysis. Similar analyses can be conducted for other blood cells including red blood cells and white blood cells.

In one aspect, the invention may comprise hemostatic agents useful in the treatment of vascular trauma, which include a sono-responsive material. These hemostatic agents can take the form of a medical device, a pharmaceutical agent, a biological agent, a medical device/pharmaceutical combination or medical device/biological combination.

In one aspect, the invention comprises a method of monitoring a treatment using a sono-responsive material, where the travel, location and/or degradation of the sono-responsive material is monitored by ultrasound. For example, a sono-responsive embolic agent may be introduced into the vasculature of a target tissue, which is intended to reduce or eliminate the blood supply to a target tissue. The travel of the embolic agent may be visualized using ultrasound. Target tissues often exhibit vascular access routes that are preferred for delivery of the embolic agent.

In this manner, the embolic agent may mimic or enhance the effect of an ultrasound contrast agent; however, the embolic agents of the present invention are much larger than conventional contrast agents, and are capable producing a therapeutically beneficial embolus.

Effective treatment of a target tissue can require more than a single treatment with an embolic agent. If the embolic agent is not biodegradable and is delivered into the preferred route of vascular access to the target tissue, retreatment of the target tissue using the preferred vascular access route is very difficult or impossible. However, a biodegradable embolic agent will allow retreatment, at a time when degradation is substantially complete. A sono-responsive biodegradable embolic agent may be monitored from time-to-time using ultrasound. Thus, an appropriate time to retreat the target tissue using the same vascular access route may be identified, for example, of the embolic agent has degraded and has minimal echogenic signal or is no longer detectable by ultrasound. In one embodiment retreatment would occur after at least 25% of the embolic agent has degraded, more preferably after at least 50% of the embolic agent has degraded, and still more preferably after at least 90% of the embolic agents has degraded, as determined by a change in echogenic signal.

In some embodiments, this treatment and monitoring method may be used to treat, monitor, and/or retreat liver tumors, hypervascularized tumors, malignant or benign tumors, kidney tumors, pancreatic tumors, lung tumors, brain tumors, gastric tumors, intestinal tumors, rectal tumors, colorectal tumors, ocular tumors, esophageal tumors, splenic tumors, uterine tumors, ovarian tumors, leiomyoma, hyperplastic tissue, hypertrophic tissue, or enlarged prostates. Other tissues which may be treated in this manner may include tissues requiring augmentation for cosmetic purposes, tissues requiring augmentation to treat a medical condition, and stomach tissue responsible for releasing hormones, chemicals or messengers that regulate hunger or satiation.

### EXAMPLES

The present invention is described with reference to the following Examples. These Examples are provided for the purpose of illustration only.

Example 1—Collagen coated PLGA showed significant platelet binding

Uncoated PLGA particles and PLGA particles coated with collagen were challenged with platelet-rich-plasma under high force/shear conditions. Platelet binding to the particles was determined using a fluorescently-labeled platelet specific antibody (anti-CD61). Particle fluorescence was also evaluated with a fluorescently-labeled isotype control antibody.

Non-coated PLGA particles visible with confocal imaging showed limited fluorescence indicated limited platelet binding. PLGA particles coated with collagen showed significant platelet binding. This result is not surprising in view of Applicant's prior work in this area.

Example 2—PLGA particles are hyperechoic.

Unmodified polymer particles with a density approximately 1.3 g/ml were evaluated using a phantom comprising ultrasound gel held within a cylindrical conduit. Unlike the spherical particles in other examples, these particles were substantially cylindrical, having a length of about 200 microns, and a diameter of about 80 microns. The particles demonstrated hyperechoic properties when examined by B-mode ultrasound. Movement of the particles induced by manipulating the conduit could be detected using B-mode ultrasound.

Example 3—Treated PLGA particles are hyperechoic

Hyperechoic PLGA particles were made spherical and treated with different chemical agents to enhance wettability and thereby enhance blood cell and platelet binding ability. The microspheres were evaluated using a phantom comprising ultrasound gel held within a cylindrical conduit. All treated particles demonstrated hyperechoic properties when examined by B-mode ultrasound. Movement of the particles induced by manipulating the conduit could be detected using B-mode ultrasound.

Example 4—Hyperechoic PLGA particles were made spherical and treated with various chemical agents to enhance wettability. The microspheres were delivered through conduits of varying diameter and passage compared to polymer microspheres that were less wettable. More wettable polymer microspheres resulted in smoother travel of the polymer microspheres through the conduit. Less wettable polymer microspheres resulted in slowed travel of the microspheres through the conduit, or in some instances, an inability to pass through the conduit.

Example 5—Hyperechoic PLGA polymer microspheres were delivered by catheter into the vasculature supplying prostatic tissue. Evaluation of the tissue pre- and immediately post-delivery using B-mode ultrasound demonstrated hyperechoic areas in the prostatic tissue post-delivery.

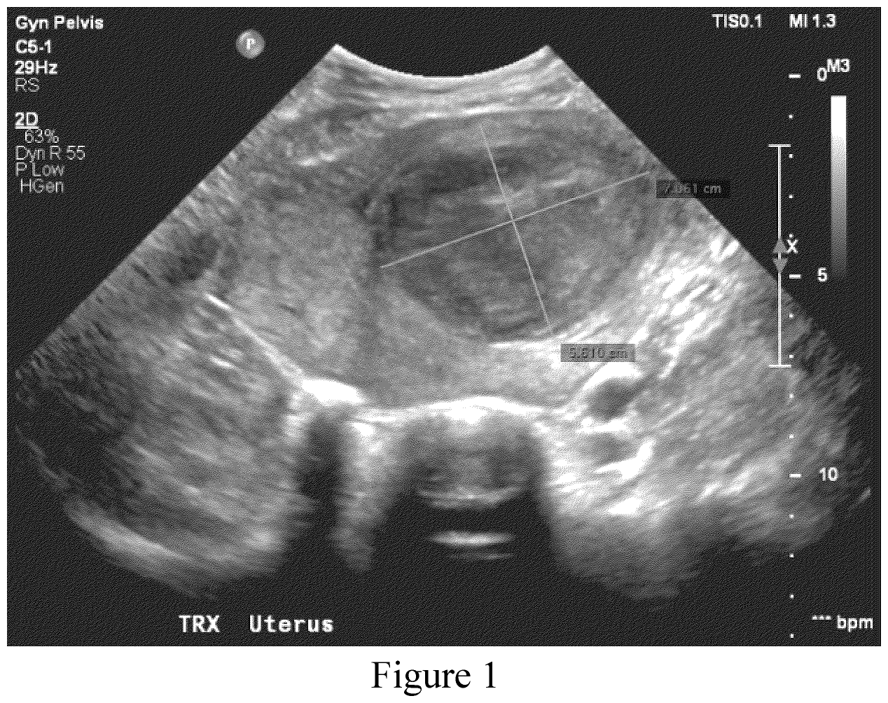

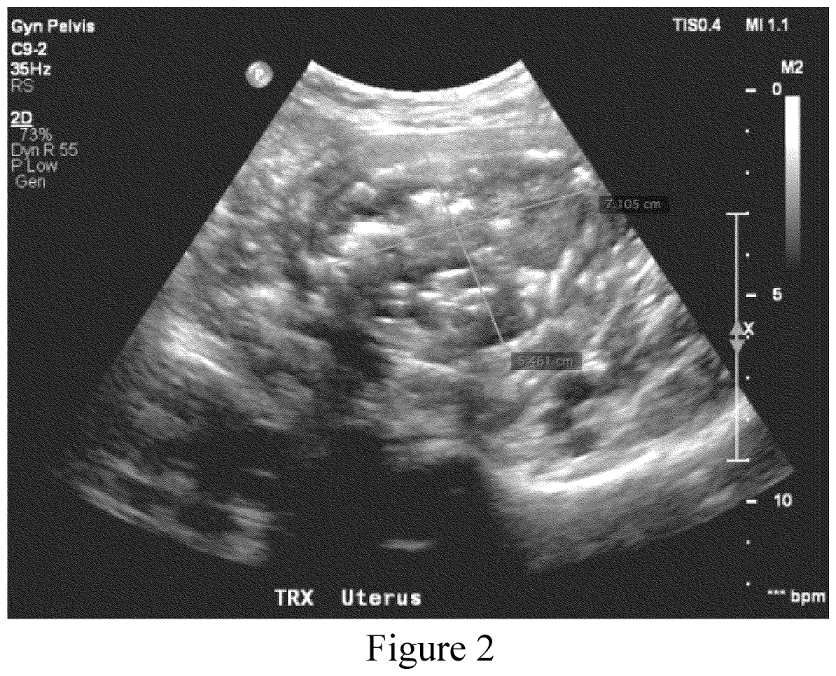

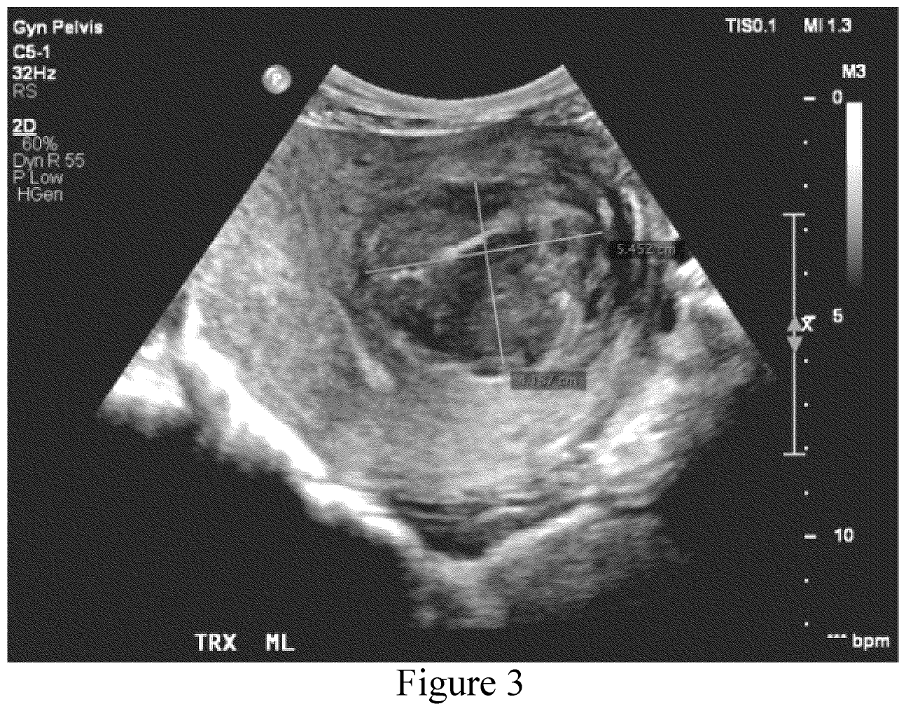

Example 6—Hyperechoic PLGA polymer microspheres were delivered by catheter into the vasculature supplying uterine tissue. Evaluation of the tissue pre- and post-delivery using B-mode ultrasound demonstrated hyperechoic areas in the uterine tissue post-delivery. As shown in FIG. 1, prior to embolization, a fibroid is faintly visible, marked by intersecting lines indicating transverse dimensions of the fibroid. FIG. 2 is a sonogram 24 hours post-embolization. Areas within the fibroid are clearly hyperechoic, indicating that the embolic agent has produced an embolus in those locations. FIG. 3 is a sonogram taken 30 days post post-embolization. The hyperechoic areas are not as bright, indicating that the embolic agent has degraded, but it is still slightly more hyperechoic than pre-embolization. The fibroid has decreased in size as a result of the embolic treatment.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “up to”, “at least”, “greater than”, “less than”, “more than”, “or more”, and the like, include the number(s) recited and such terms refer to ranges that can be subsequently broken down into sub-ranges as discussed above.

1. A method of treating a patient in need of treatment, comprising the steps of:
(a) introducing a sono-responsive embolic agent into the patient; and
(b) monitoring the travel and/or position of the sono-responsive embolic agent by ultrasound.

2. The method of claim 1 wherein the sono-responsive embolic agent has a density greater than about 1.1 g/ml, or greater than about 1.2 g/ml, or greater than about 1.3 g/ml, or less than about 0.95 g/ml, or less than about 0.90 g/ml, or less than about 0.85 g/ml.

3. The method of claim 2 wherein the sono-responsive embolic agent comprises a glass, ceramic, polymer, metal, alloy, elastomer, pyrolytic carbon and plastics, or combinations thereof.

4. The method of claim 3 wherein the sono-responsive embolic agent comprises a polymer comprising polyvinyl alcohol (PVA); polyglycol; polyglyconate; polyetheretherketone; polyacetal; polystyrene; polycarbonate; polylactide; polyglycolide; lactide-glycolide copolymers; polycaprolactone; lactide-caprolactone copolymers; hydroxyapatite; polyhydroxybutyrate; polyalkylcyanoacrylates; polyanhydrides; polyorthoesters; polysaccharides; dextrans; starches; methyl methacrylate; methacrylic acid; hydroxylalkyl acrylates; hydroxylapatite; hydroxylalkyl methacrylates; methylene glycol dimethacrylate; acrylamide; bisacrylamide; cellulose-based polymers; polyethylene; polyethylene terephthalate; ethylene glycol polymers and copolymers; oxyethylene and oxypropylene polymers; trimethylenecarbonate; polyvinyl acetate; polyvinylpyrrolidone and polyvinylpyridine; alone or in combination.

5. The method of claim 4 wherein the polymer is biodegradable.

6. The method of claim 1 wherein the treatment is a treatment for vascular trauma, an incision, a puncture wound or other damaged blood vessels.

7. The method of claim 1 wherein the treatment is a treatment for hypervascular tumors, aneurysms, arteriovenous malformations, endoleaks, varicoceles, peripheral vascular disease, benign prostatic hyperplasia, or obesity.

8. The method of claim 1 wherein ultrasound monitors travel of the sono-responsive embolic agent prior to and including formation of an embolus.

9. The method of claim 8 wherein the ultrasound monitoring occurs in real-time.

10. The method of claim 1 wherein ultrasound monitors degradation of an embolus after formation.

11. The method of claim 1 wherein the embolic agent is modified to enhance blood cell binding.

12. The method of claim 11, wherein the embolic agent is modified by exposure to radiation or gamma radiation or treatment with a chemical agent or enzyme.

13. The method of claim 12 wherein the chemical agent comprises hydrogen peroxide, an inorganic or organic base, ethanol, an inorganic or organic acid.

14. The method of claim 1 wherein the sono-responsive embolic agent is a particle or microsphere, having a dimension larger than about 10 microns.

15. The method of claim 10 comprising the further step of retreatment with an embolic agent following degradation of the embolus, which degradation is determined by a change in echogenic signal from ultrasonic imaging of the embolus.

16. The method of claim 15 wherein the retreatment step occurs after a minimal echogenic signal is detected within target vasculature.

17. The method of claim 15 wherein the retreatment step occurs after at least 25% of the embolic agent has degraded, or after at least 50% of the embolic agent has degraded, or after at least 90% of the embolic agent has degraded.

18. The method of claim 1, wherein the treatment or retreatment is for a liver tumor, hypervascularized tumor, malignant or benign tumor, kidney tumor, pancreatic tumor, lung tumor, brain tumor, gastric tumor, intestinal tumor, rectal tumor, colorectal tumor, ocular tumor, esophageal tumor, splenic tumor, uterine tumor, ovarian tumor, leiomyoma, hyperplastic tissue, hypertrophic tissue, or enlarged prostate.

19. The method of claim 1, wherein the treatment or retreatment is to treat a tissue requiring augmentation for cosmetic purposes, tissue requiring augmentation to treat a medical condition, or stomach tissue responsible for releasing hormones, chemicals or messengers that regulate hunger or satiation.

20. A method of monitoring a treatment involving the use of a sono-responsive embolic agent comprising the step of determining the travel, location and/or degradation of the sono-responsive embolic agent by ultrasound.
